(19) United States
(12) Patent Application Publication
(10) Pub. No.: US 2008/0319388 A1
(43) Pub. Date: Dec. 25, 2008

(54) DEVICE DELIVERY SYSTEM WITH BALLOON-RELATIVE SHEATH POSITIONING

(76) Inventors: David Slattery, Kinvara (IE); Damian Kelly, Loughrea (IE); Michael Gilmore, Loughrea (IE); Mark Steckel, Sharon, MA (US)

Correspondence Address: RISSMAN JOBSE HENDRICKS & OLIVERIO, LLP, 100 Cambridge Street, Suite 2101, BOSTON, MA, 02114, US

(21) Appl. No.: 11/766,198

(22) Filed: Jun. 21, 2007

(51) Int. Cl. A61M 25/10

(52) U.S. Cl. 604/103.05

(57) ABSTRACT

A medical device delivery system includes a self-expanding medical device mounted on a balloon portion of a catheter. A sheath is provided around the medical device to hold the device in place with the device staying in a compressed state. A series of perforations or an initial cut is provided in the sheath at a predetermined location, or within a predetermined range of locations, on the circumference of the sheath. The predetermined location or range of locations are determined with respect to the folds of the balloon portion.

### RELATED APPLICATIONS

1. Field of the Invention

The present invention relates to a delivery system and method for deployment of a medical device, e.g., a self-expanding vascular device, in the vasculature of a patient. More particularly, a delivery system having a sheath with portions specifically positioned relative to a balloon positioned therein is described.

2. Background of the Invention

As is known, treatment of vascular blockages due to any one of a number of conditions, such as arteriosclerosis, often comprises balloon dilatation and treatment of the inner vessel wall by placement of a stent. These stents are positioned to prevent restenosis of the vessel walls after the dilatation. Other devices, often referred to as drug eluting stents, are now being used to deliver medicine to the vessel wall to also help reduce the occurrence of restenosis.

These stents, i.e., tubular prostheses, typically fall into two general categories of construction. The first category of prosthesis is made from a material that is expandable upon application of a controlled force applied by, for example, a balloon portion of a dilatation catheter upon inflation. The expansion of the balloon causes the compressed prosthesis to expand to a larger diameter and then left in place within the vessel at the target site. The second category of prosthesis is a self-expanding prosthesis formed from, for example, shape memory metals or super-elastic nickel-titanium (NiTi or Nitinol) alloys, that will automatically expand from a compressed or restrained state when the prosthesis is advanced out of a delivery catheter and into the blood vessel.

Some known prosthesis delivery systems for implanting self-expanding stents include an inner lumen upon which the compressed or collapsed prosthesis is mounted and an outer restraining sheath that is initially placed over the compressed prosthesis prior to deployment. When the prosthesis is to be deployed in the body vessel, the outer sheath is moved in relation to the inner lumen to “uncover” the compressed prosthesis, allowing the prosthesis to move to its expanded condition. Some delivery systems utilize a “push-pull” type technique in which the outer sheath is retracted while the inner lumen is pushed forward. Still other systems use an actuating wire that is attached to the outer sheath. When the actuating wire is pulled to retract the outer sheath and deploy the prosthesis, the inner lumen must remain stationary, preventing the prosthesis from moving axially within the body vessel.

There have been, however, problems associated with these delivery systems. For example, systems that rely on a “push-pull design” can experience movement of the collapsed prosthesis within the body vessel when the inner lumen is pushed forward. This movement can lead to inaccurate positioning and, in some instances, possible perforation of the vessel wall by a protruding end of the prosthesis. Systems that utilize an actuating wire design will tend to move to follow the radius of curvature when placed in curved anatomy of the patient. As the wire is actuated, tension in the delivery system can cause the system to straighten. As the system straightens, the position of the prosthesis changes because the length of the catheter no longer conforms to the curvature of the anatomy. This change of the geometry of the system within the anatomy can also lead to inaccurate prosthesis positioning.

Other delivery systems are known where a self-expanding stent is kept in its compressed state by a sheath positioned about the prosthesis. A balloon portion of the delivery catheter is provided to rupture the sheath and, therefore, release the prosthesis. As shown in U.S. Pat. No. 6,656,213, the stent may be provided around the balloon, with the sheath around the stent, that is, the balloon, stent, and sheath are co-axially positioned, such that expansion of the balloon helps to expand the self-expanding stent as well as rupture the sheath. In other embodiments, the balloon is outside the stent and the sheath is around both the balloon and the stent.

To facilitate the rupturing of the sheath, it is further known to provide perforations in the sheath. The intention of the perforations is to make the rupturing or separation of the sheath easier upon expansion of the balloon. While the perforations may help to control the rupturing of the sheath by providing a “weak” portion, the dynamics of sheath rupturing are still not well controlled.

There is, therefore a need for a mechanism to reliably deliver a self-expanding stent, enclosed in a sheath, with repeatable and known operating characteristics.

### SUMMARY OF THE INVENTION

In one embodiment, a delivery system comprises a catheter having a distal end and a proximal end; a balloon, in a deflated condition, positioned at the distal end of the catheter, the balloon comprising at least two wing portions wrapped about the distal end of the catheter, and a sheath positioned about the balloon, wherein the sheath comprises a weakened portion located on the positioned sheath in a predetermined relation to the at least two wing portions of the balloon.

The weakened portion of the positioned sheath may comprise a plurality of substantially linearly arranged perforations oriented substantially parallel to a longitudinal axis of the sheath.

The weakened portion of the positioned sheath may be located at a position where a total force exerted by expansion of the at least two wing portions against the positioned sheath, upon inflation of the balloon, is at its greatest.

The weakened portion of the positioned sheath may be located at a position that is approximately equidistant between sequentially adjacent circumferential points where the at least two wings press against the positioned sheath as the balloon is inflated.

Upon inflation of the balloon, in one embodiment, each wing of the at least two wings presses against the positioned sheath at a respective wing pressure location about the circumference of the sheath; and the weakened portion of the positioned sheath is located at a position that is approximately half the distance, around the circumference, between adjacent wing pressure locations.

The weakened portion of the positioned sheath may comprise an initial cut in the sheath extending proximally a predetermined distance from a distal edge of the sheath.

In yet another embodiment, the predetermined location of the weakened portion may be within 20% of a midpoint between sequentially adjacent circumferential points where the at least two wings press against the positioned sheath as the balloon is inflated.

In one embodiment, the delivery system may comprise: a self-expanding medical device positioned at the distal end of the catheter, wherein the self-expanding medical device is maintained in a compressed state by the positioned sheath.

In one embodiment the balloon is a dual-wing balloon having first and second wings, each wing having a respective wing-tip portion and a wing-base portion, wherein the balloon is wrapped about the catheter in a bi-fold orientation, and wherein the opening in the sheath is located between the wing-tip portion of the first wing and the wing-base portion of the second wing.

n another embodiment the balloon is a dual-wing balloon having first and second wings, 1 each wing having a respective wing-tip portion and a wing-base portion, and wherein the balloon is wrapped about the catheter in a U-fold orientation, and wherein the opening in the sheath is located between the wing tip of the first wing and the wingtip of the second wing.

In another embodiment the balloon is a tri-wing balloon having three wings, each wing having a respective wingtip portion and wing base portion, wherein the balloon is wrapped about the catheter such that a wingtip portion of a first wing is folded toward a wingbase portion of a next adjacent wing, and wherein the opening in the sheath is located between the wingtip portion of the first wing and the wingbase portion of the next adjacent wing.

In yet another embodiment a system comprises: a catheter having a distal end and a proximal end; a balloon, in a deflated condition, positioned at the distal end of the catheter and wrapped about the distal end of the catheter, the balloon comprising at least two wing portions; a medical device, having a compressed state and an expanded state, positioned about the balloon; and a sheath, comprising sheath material, positioned about the medical device to hold the medical device in the compressed state, the sheath material comprising a predetermined sheath portion, wherein the predetermined sheath portion is located at a position as a function of positions of the at least two wing portions of the balloon.

The balloon may be one of: a bi-wing structure with only two wings; and a tri-wing structure with three wings.

In yet another embodiment a method of providing an ostial protection device delivery system comprises: providing a catheter having a distal end and a proximal end; positioning a deflated balloon at the distal end of the catheter, the balloon comprising wing portions; wrapping the wing portions about the distal portion of the catheter so as to facilitate inflation of the balloon; providing an ostial protection device about the deflated balloon; positioning an elongate tubular sheath, the sheath having a proximal end and a distal end oriented with the catheter, the sheath comprising a predefined sheath portion, about the ostial protection device and the balloon to hold the ostial protection device in a compressed state; and orienting the predefined sheath portion in a predetermined relationship as a function of locations of the folded wing portions of the balloon.

### BRIEF DESCRIPTION OF THE DRAWINGS

The above and further advantages of the present invention may be better understood by referring to the following description in conjunction with the accompanying drawings in which:

FIG. 1A is a view of a device delivery system;

FIG. 1B is a cross-sectional view of the device delivery system along line 1B-1B as shown in FIG. 1A;

FIGS. 2A and 2B are views of a portion of a device delivery system;

FIG. 3 is a cross-sectional view of a dual-wing PTCA balloon;

FIG. 4 is a cross-sectional view of the dual-wing PTCA balloon of FIG. 3 in a bi-folded configuration and wrapped within a sheath;

FIG. 5 is a cross-sectional view of the partially expanded PTCA balloon of FIGS. 3 and 4;

FIG. 6 is a perspective view of an initial cut positioned on a sheath;

FIG. 7 is a cross-sectional view of a tri-wing PTCA balloon;

FIG. 8 is a cross-sectional view of the tri-wing PTCA balloon of FIG. 7 in a tri-folded configuration and wrapped within a sheath;

FIG. 9 is a cross-sectional view of a dual-wing PTCA balloon in a U-fold configuration and wrapped within a sheath;

FIG. 10 is a method of placing a sheath split with respect to an orientation of a balloon placed within;

FIG. 11 is an alternate method of loading a device on a delivery system and orienting the splits in the sheath with a balloon; and

FIG. 12 is a perspective view of the dual-wing PTCA balloon of FIG. 3.

### DETAILED DESCRIPTION

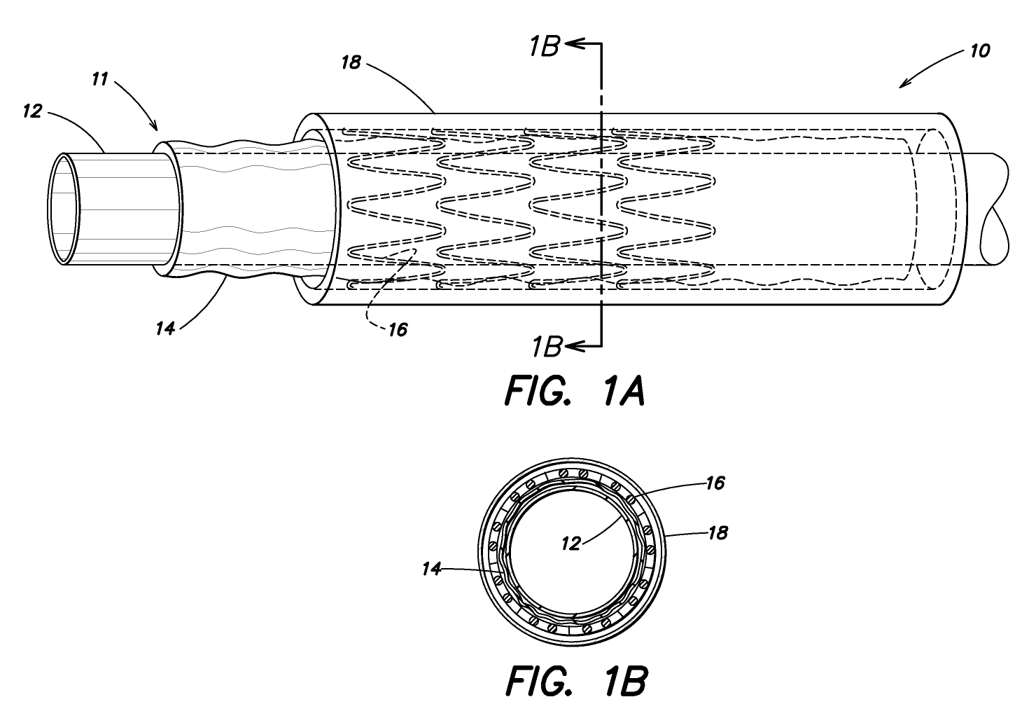

A medical device delivery system, as shown in FIG. 1A, includes a delivery catheter 12 with a balloon 14 positioned at, or enclosing, a distal end 11 of the catheter 12. As is known, a lumen is provided to inflate the balloon 14 as necessary during the procedure to deliver a device 16, for example, a stent, that is placed at the distal end of the catheter 12 and around the balloon 14. As per the present discussion, the device 16 is a self expanding device and, therefore, a sheath 18 is also disposed at the distal end 11 of the catheter 12 so as to enclose the device 16 and the balloon 14. The sheath 18 is attached to the catheter 12 at some point proximal to the distal end 11 of the catheter 12.

A cross section of the system 10, along line 1B-1B, is presented in FIG. 1B. As shown, the sheath 18 surrounds the stent or device 16 and the balloon 14 positioned on the catheter 12.

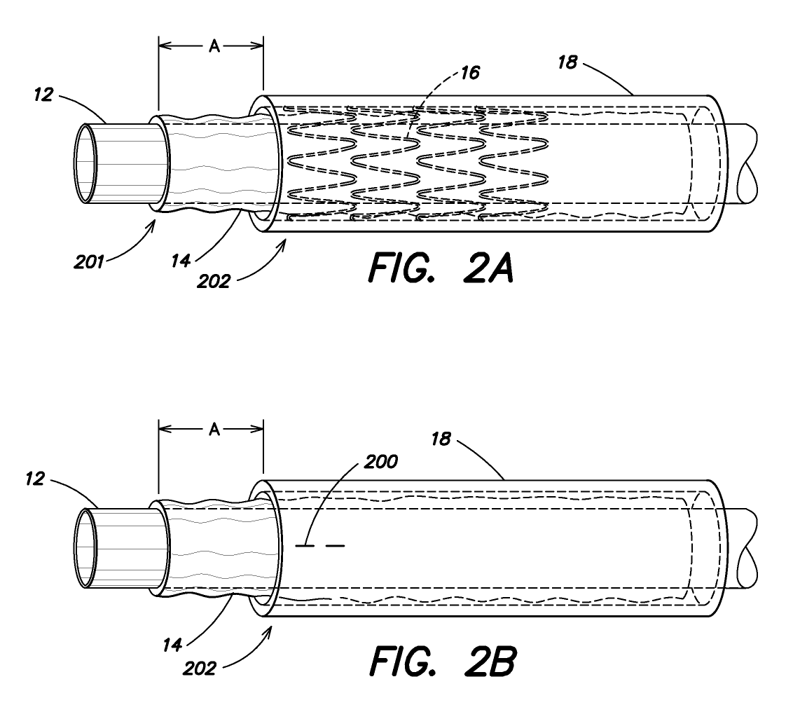

Referring to FIG. 2A, a simpler representation of the system 10 of FIG. 1A, is shown where a distal end 202 of the sheath 18 is positioned a distance A proximally from a distal end 201 of the balloon 14. Placing the distal edge 202 of the sheath 18 a predetermined distance proximal to the distal end 201 of the balloon 14 allows for maximum effectiveness of the balloon 14 with respect to rupturing of the sheath 18. Referring now to FIG. 2B, corresponding to FIG. 2A, with the device 16 removed for clarity, a perforation 200 is provided in the sheath 18. The perforation 200 is shown here at the distal end 202 of the sheath 18. The perforation 200 facilitates the separation or rupture of the sheath 18 as the balloon 14 is expanded. The perforation 200 may comprise, in one embodiment, one or more discontinuous slits in the sheath material 18. Here, a slit does not involve the removal of sheath material.

Alternatively, the sheath 18 may be created by weakening a portion of the sheath 18 by chemical and/or mechanical means. Still further, the perforation 200 may comprise one or more holes, where each hole is created by the removal of sheath material. While the perforation 200 is shown here at the distal end of the sheath 18, of course, one of ordinary skill in the art would understand that were the sheath 18 to be connected to the catheter 12 at the distal end of the sheath 18, then the perforation 200 may be positioned at a proximal end of the sheath 18. Further, more than one perforation 200 may be provided, for example, one at each of the proximal and distal ends of the sheath 18, respectively.

As an alternative to the perforation 200, a single initial cut 602, as shown in FIG. 6, may be implemented.

The sheath is made from a material having a grain, or fibers, that can be longitudinally oriented, for example, PTFE. In general, the sheath 18, upon expansion of the balloon 14, will tear along the perforation 200 or initial cut 602 in substantially a straight line following a longitudinal axis of the sheath 18 as defined, generally, by the catheter 12.

The expansion of the balloon causes the sheath to rupture. Once the sheath ruptures, the stent expands and is released into the vessel. The balloon pressure that will cause a sheath 18 to split, however, is not consistent in previously known systems. Tests have shown that the sheaths from a batch of balloon-mounted systems will not always split at the same balloon pressure.

Experiments were conducted by the present inventors, using an external polymer sheath 18, made from PTFE, and a PTCA balloon catheter. In the test setup, the PTCA balloon catheter included a 2.0 mm by 30 mm nylon balloon 14. The particular type of balloon 14 that was used in these experiments exhibited semi-compliant behavior, in that it has a compliance of approximately 5% at 2 mm with 6 atmospheres of nominal pressure, i.e., the balloon diameter ranges from 1.9 mm to 2.1 mm at 6 atmospheres. The external polymer sheath 18 was provided in two sizes: a) 0.043 inches inner diameter by 0.002 inches wall thickness; and b) 0.047 inches inner diameter by 0.002 inches wall thickness. The sheath was positioned substantially as shown in FIGS. 1A and 1B. Further, an initial cut was placed at the distal end of the sheath 18.

Five samples of each of sheath type a) and sheath type b) were externally loaded onto the 2.0 mm by 30 mm PTCA balloon catheter. The balloon was inflated in one atmosphere intervals to a pressure until the polymer sheath fully split, i.e., the sheath split along the full length of the 30 mm PTCA balloon. As shown in Table 1 below, for sheath type (a) the balloon pressure needed to fully split the sheath ranged from 4 to 8 atmospheres. The sheath of type (b) exhibited a full split with balloon pressures that ranged from 7 to 18 atmospheres.

TABLE 1

| No. | Sheath | Split (atm) |
|---|---|---|
| 1 | (a) | 4 |
| 2 | (a) | 4 |
| 3 | (a) | 8 |
| 4 | (a) | 6 |
| 5 | (a) | 6 |
| 1 | (b) | 9 |
| 2 | (b) | 9 |
| 3 | (b) | 12 |
| 4 | (b) | 7 |
| 5 | (b) | 18 |

This inconsistency in the balloon pressure required to fully split the polymer sheath appears to hinder the effectiveness of the sheath for delivery of a device. The wide range of balloon pressure values required to fully split the sheath renders a construction substantially as represented in FIG. 1A too variable to validate and subsequently too variable to use in everyday procedures.

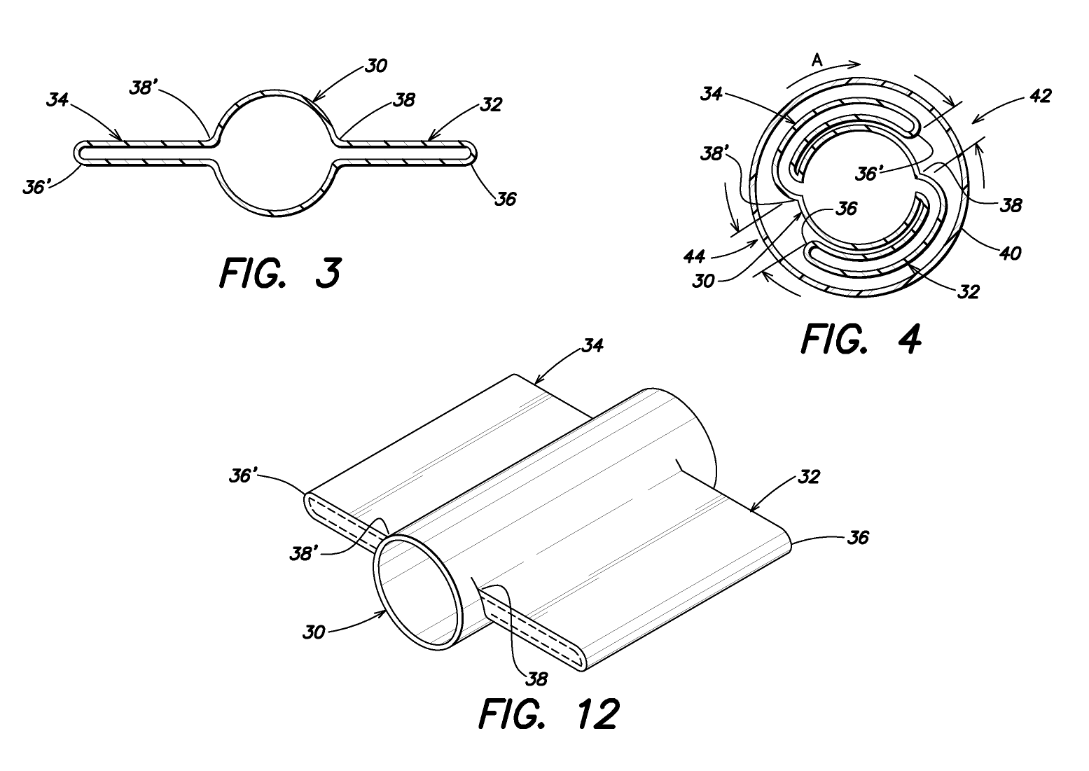

The present inventors recognized that the bi-folded wings of a PTCA catheter balloon could be used to aid in better controlling the splitting dynamics of the sheath. A deflated PTCA catheter balloon 30, shown in a perspective view in FIG. 12 and in cross section in FIG. 3, includes, when the PTCA balloon 30 is vacuumed, two substantially equal wings 32, 34. Each wing has a wing tip 36 and a wing base 38. It should be noted that the catheter 12 and stent 16 are not shown in FIGS. 3 and 4 for clarity although one of ordinary skill in the art would certainly understand the positioning of these components in a system according to the present disclosure.

Referring to FIG. 4, the PTCA balloon 30, once mounted on the delivery system, is folded such that the wings 32, 34 “wrap-around” the body of the balloon 30 in such a way so as to not interfere with each other as the balloon 30 is inflated, i.e., a “wrap bi-fold” orientation. In general, a wing tip portion 36′ of the wing 34 is folded along a circumferential direction A (shown by arrow) toward the base portion 38 of the wing 32. Similarly, the wing tip portion 36 of the wing 32 is folded toward the wing base portion 38′ of the wing 34, continuing in the direction A. Looking along the axis of the system, as shown in FIG. 4, the results of the folds of the balloon in this fashion are similar to a child's pinwheel. A sheath 40 is then provided over the folded balloon, and the device 16 (not shown) to keep the device 16 in a compressed state.

The present inventors have observed that placement of the perforation 200 or split 602 to take advantage of the mechanical leverage provided from the folded wings 32, 34 of the balloon 30 will aid in establishing a consistent and repeatable splitting of the sheath at a specific pressure, or relatively narrow range of pressures, of the balloon. In known systems, the split or perforation on the sheath were randomly placed, irrespective of any geometry of the balloon around which the sheath was disposed.

As found by the inventors of the present invention, there is an optimum area or areas on the circumference of the sheath at which to place the perforation 200 (running longitudinally) or initial cut 602. These locations around the circumference are determined by the folded balloon.

Referring to FIG. 4 a sheath 40 has been provided around a dual-wing balloon 30 in a wrap bi-folded configuration. Two placement areas 42, 44 along the circumference of the sheath 40 are defined. Placing the perforation 200 or cut 602 within at least one of these placement areas optimizes the tearing or rupturing of the sheath 40. These two areas 42, 44 are defined or predetermined with respect to the orientation of the folded balloon.

When the perforation 200 or initial cut 602 is placed anywhere within one of the areas 42, 44, the sheath 40 will split at a uniform and consistent and repeatable pressure of the balloon. It should be noted that one initial cut or perforation in either of the areas 42, 44 is sufficient to initiate the full split of the sheath 40. It has been observed, however, that a split or perforation may be placed in each of the areas 42, 44 to facilitate separation of the sheath 40.

A second set of experiments was performed where sheaths, with the same construction as those previously described, are provided around the PTCA balloon except that the perforation or initial cuts are placed in one of the areas 42, 44, i.e., relative to the orientation of the balloon 30. Once again, the balloon is inflated in increments of 1 atmosphere. As shown in Table 2, the balloon pressure necessary to fully split the sheath 40 was repeatedly 5 atmospheres.

TABLE 2

| No. | Sheath | Split (atm) |
|---|---|---|
| 1 | (a) | 5 |
| 2 | (a) | 5 |
| 3 | (a) | 5 |
| 4 | (a) | 5 |
| 5 | (a) | 5 |
| 1 | (b) | 5 |
| 2 | (b) | 5 |
| 3 | (b) | 5 |
| 4 | (b) | 5 |
| 5 | (b) | 5 |

The specific placement of the initial cut 602 or perforation 200 with respect to the folded geometry or orientation of the balloon provides consistent and repeatable sheath splitting performance. The repeatability and consistency of obtaining a full split provides an advantage with respect to using a delivery system with a balloon expandable sheath to deliver a self expanding medical device.

Thus, the present inventors have recognized that the folds or wings 32, 34 of the PTCA balloon 30 play a role in splitting the sheath 40, due to the placement of the split 602 or perforation 200. Further, optimum positions about the circumference of the sheath can be predetermined as a function of the balloon's placement and folded geometry about the catheter.

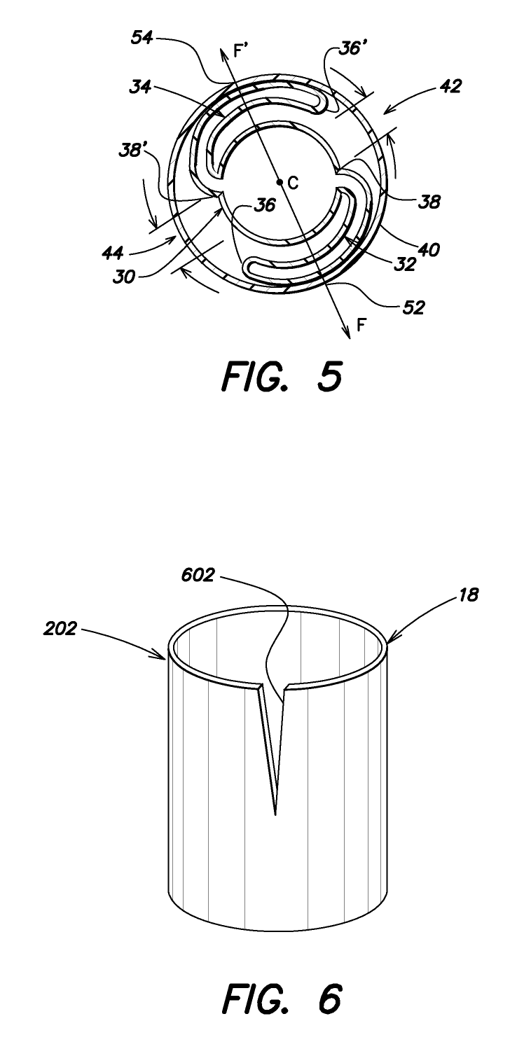

Referring to FIG. 5, the placement areas 42, 44 can be defined as those locations around the circumference of the sheath 40 at which the resultant force exerted by the wings 32, 34, against the sheath as the balloon is inflated, is at a maximum. It can be considered that the balloon 30 expands symmetrically from its center C as it is being inflated. The wings 32, 34 exert, respectively, forces F and F′, against the sheath 40 at points 52, 54, respectively. The cumulative effect of the forces of the wings 32, 34 against the sheath 40 is maximized in the two placement areas 42, 44. Thus, placing a perforation or an initial cut in either or both of the placement areas 42, 44 provides for a repeatable and consistent splitting of the sheath 40 at a known pressure.

The placement areas 42, 44 located about the circumference of the sheath 40 may be considered to be defined as located generally halfway between circumferentially adjacent points where the balloon wings 32, 34 exert a respective force against the sheath 40 upon inflation of the balloon. The placement areas 42, 44, in one embodiment are located along the circumference of the sheath within a portion of the circumference that is in a range of 40-60% of the distance between the points 52, 54.

Alternatively, the location of the placement areas 42, 44 may be described as being located between a wing tip 36 and a wing base 38 of adjacent wings of the balloon. As shown in FIG. 5, due to the bi-fold of the balloon 30, the wing tip portion 36′ is adjacent the wing base portion 38. The placement area 42 is, therefore, located substantially half-way between these two wing portions. Advantageously, the placement areas 42, 44 are easily discernible by viewing the folded balloon within the sheath.

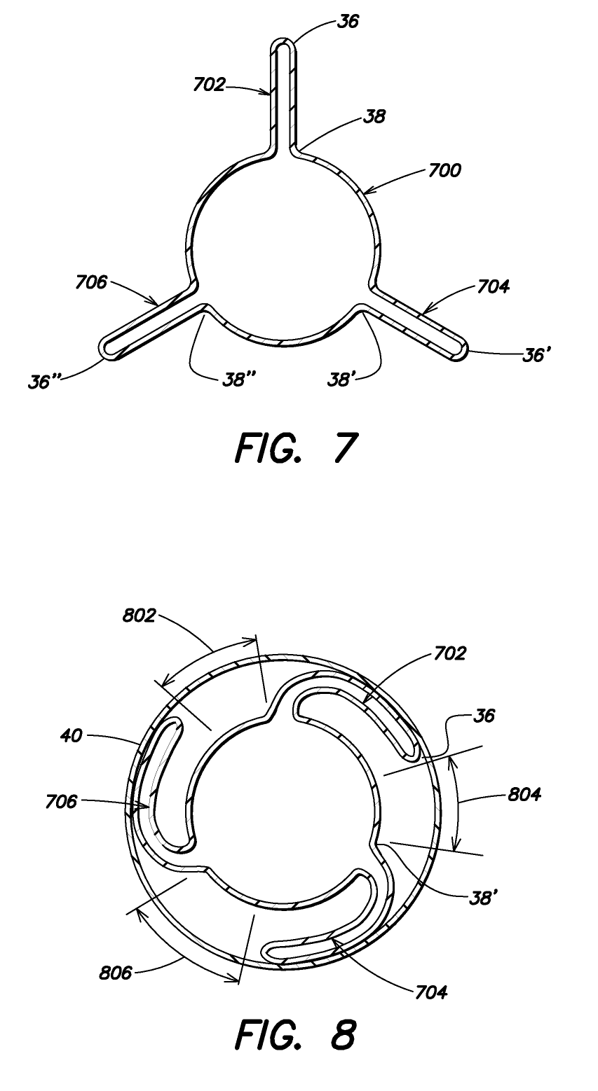

The balloon 30, as shown in FIG. 3, is of a dual-wing design. Alternatively, a balloon 700 of a tri-wing design, as shown in cross-section in FIG. 7, may be used. As shown, the balloon 700 has three wings 702, 704, 706 symmetrically disposed about the circumference of the balloon. Each of the wings has a wing tip 36 and a wing base 38.

When folded, and placed within a sheath 40, as shown in cross-section in FIG. 8, placement areas 802, 804, 806 are positioned about the circumference of the sheath 40. Similar to the foregoing description, the placement areas 804, 806 are, respectively, located between adjacent wing tip portions 36 and wing base portions 38.

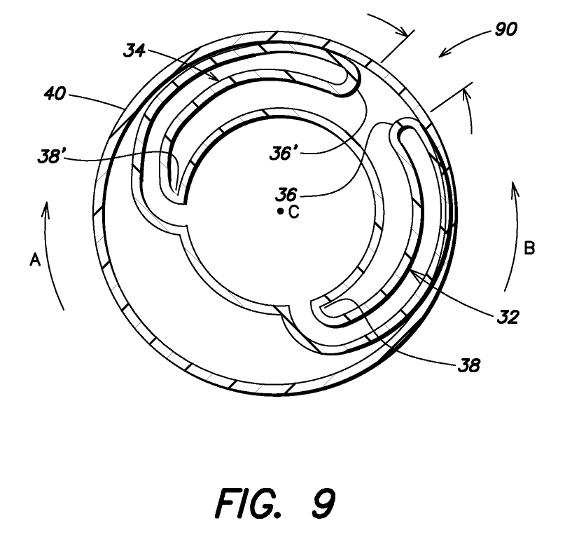

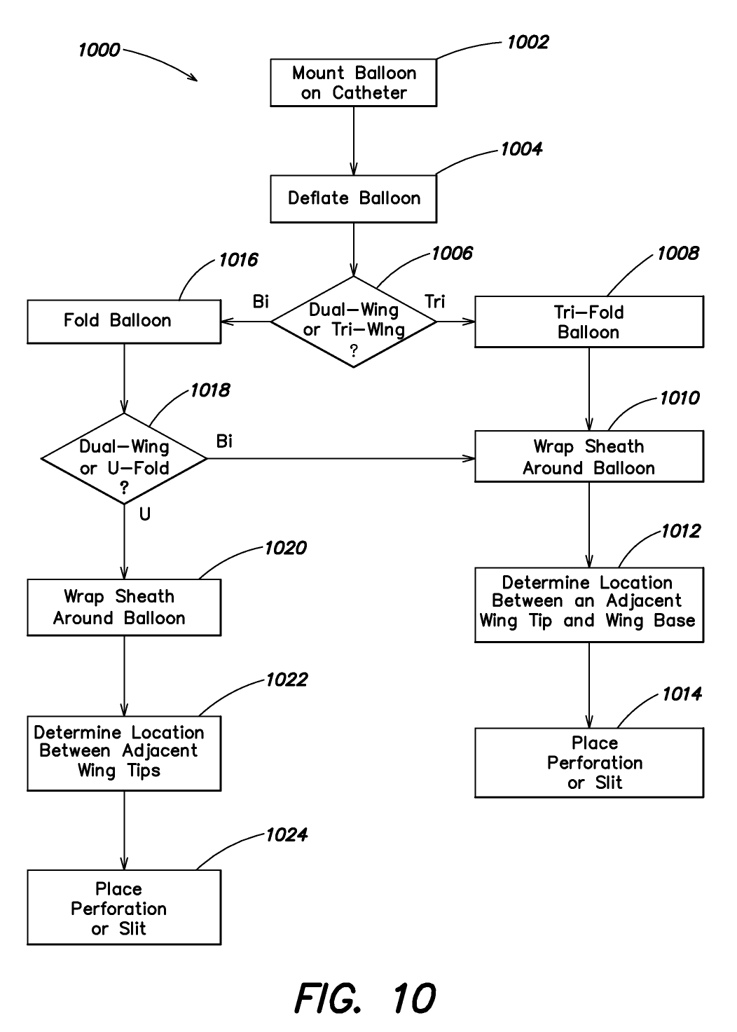

In yet another embodiment, as shown in FIG. 9, the dual-wing balloon is folded in a U-fold, where the wings 32, 34 have their respective wingtip portions 36, 36′ adjacent one another. In this configuration, the wing 34 is wrapped in the circumferential direction A (as shown by the arrow A) while the wing 32 is wrapped in an opposite circumferential direction B (as shown by the arrow B) opposite that of direction A. The placement area 90 is then located along the circumference of the sheath 40 substantially midway between the wingtip portions 36, 36′. It is expected that as the balloon is inflated in this orientation the cumulative effect of the wing portions pushing on this sheath will be maximized within the placement area 90.

A method 1000 for assembling a delivery system as described above is shown, generally, in FIG. 10. Initially, step 1002, the balloon is mounted on the catheter. For the sake of simplicity, reference to a medical device being mounted is not included in this description, however, one of ordinary skill in the art will understand where the medical device would be installed. Subsequently, step 1004, the balloon is mostly deflated, i.e., a vacuum is created within the balloon lumen. At step 1006 it has to be determined whether or not the balloon is of a dual-wing or tri-wing construction. If it is the latter, control passes to step 1008 where the balloon is folded in a tri-fold configuration. The sheath is then wrapped around the balloon, step 1010. One or more locations between an adjacent wing-tip and wing-base are then determined at step 1012. Once the location of the placement area is determined in step 1012, the perforation or slit is provided at step 1014.

Returning to step 1006, if the balloon is of a dual-wing construction then control passes to step 1016 where the balloon is folded. At step 1018 it is determined as to whether or not the balloon was folded in a bi-fold configuration or a U-fold configuration. If it is determined that is the former configuration then control passes to step 1010 and operation continues as described above. If, however, it is the U-fold configuration then, at step 1020, the sheath is wrapped around a balloon. Subsequently, step 1022, the location between adjacent wing tips about the circumference of the sheath is determined. Finally, step 1024, the perforation or slit is placed in the determined location.

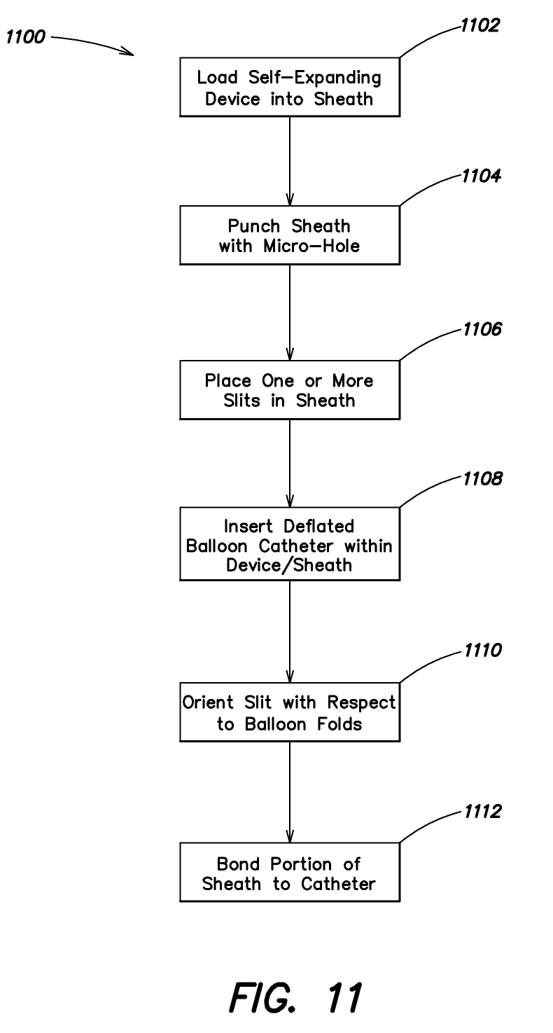

An alternate method 1100 for assembling a system in accordance with another embodiment of the present invention will now be described with respect to the flowchart shown in FIG. 11. Initially, a self-expanding device is loaded into a sheath, step 1102. A micro-hole is then punched into the sheath, step 1104, in order to facilitate the flow-through of liquid, for example, blood, as may be found in a vessel in which the device will be placed. One or more slits or perforations or holes are placed in the sheath, step 1106. A deflated balloon, with its wings folded in one of the orientations described above, is positioned on a catheter which is then inserted within the device/sheath assembly, step 1108. The previously provided slit or perforation is then oriented with respect to the balloon fold, in accordance with the previously described process, step 1110. Once aligned, a portion of the sheath is bonded to the catheter to maintain this orientation, step 1112.

It is to be understood that the present invention is not limited in its application to the details of construction and the arrangement of the components set forth in the foregoing description or illustrated in the drawings. The invention is capable of other embodiments or of being practiced or carried out in various ways. Also, it is to be understood that the phraseology and terminology employed herein are for the purpose of description and should not be regarded as limiting.

It is further appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

While the present invention has been described with respect to a bi-folded balloon, the invention is not limited to an embodiment with a balloon that only has two wings. The present invention can be implemented with any balloon having two or more wings where the initial cut or perforation are placed in the sheath with respect to those points on the sheath at which the wings of the balloon exert force against the sheath as the balloon is being inflated.

Thus, in accordance with the teachings of the present invention, the placement of one or more initial cuts or series of perforations in a sheath that is provided to constrain a self expanding device, for example, a stent prior to delivery is determined with respect to a geometry and orientation of a folded balloon around which the sheath is provided.

Although various exemplary embodiments of the present invention have been disclosed, it will be apparent to those skilled in the art that changes and modifications can be made that will achieve some of the advantages of the invention without departing from the spirit and scope of the invention. It will be apparent to those reasonably skilled in the art that other components performing the same functions may be suitably substituted.

What is claimed is:

1. A delivery system, comprising:
a catheter having a distal end and a proximal end;
a balloon, in a deflated condition, positioned at the distal end of the catheter, the balloon comprising at least two wing portions wrapped about the distal end of the catheter, and
a sheath positioned about the balloon, wherein the sheath comprises a weakened portion located on the positioned sheath in a predetermined relation to the at least two wing portions of the balloon.

2. The delivery system of claim 1, wherein the weakened portion of the positioned sheath comprises a plurality of substantially linearly arranged perforations oriented substantially parallel to a longitudinal axis of the sheath.

3. The delivery system of claim 1, wherein:
the weakened portion of the positioned sheath is located at a position where a total force exerted by expansion of the at least two wing portions against the positioned sheath, upon inflation of the balloon, is at its greatest.

4. The delivery system of claim 1, wherein:
the weakened portion of the positioned sheath is located at a position that is approximately equidistant between sequentially adjacent circumferential points where the at least two wings press against the positioned sheath as the balloon is inflated.

5. The delivery system of claim 1, wherein:
upon inflation of the balloon, each wing of the at least two wings presses against the positioned sheath at a respective wing pressure location about the circumference of the sheath; and
the weakened portion of the positioned sheath is located at a position that is approximately half the distance, around the circumference, between adjacent wing pressure locations.

6. The delivery system of claim 1, wherein the weakened portion of the positioned sheath comprises an initial cut in the sheath extending proximally a predetermined distance from a distal edge of the sheath.

7. The delivery system of claim 1, wherein the predetermined location of the weakened portion is within 20% of a midpoint between sequentially adjacent circumferential points where the at least two wings press against the positioned sheath as the balloon is inflated.

8. The delivery system of claim 1, further comprising:
a self-expanding medical device positioned at the distal end of the catheter,
wherein the self-expanding medical device is maintained in a compressed state by the positioned sheath.

9. The delivery system of claim 8, wherein:
the self-expanding medical device is positioned between the balloon and the sheath.

10. The delivery system of claim 1, wherein the weakened portion of the sheath comprises an opening in the sheath.

11. The delivery system of claim 10, wherein:
the balloon is a dual-wing balloon having first and second wings, each wing having a respective wing-tip portion and a wing-base portion,
wherein the balloon is wrapped about the catheter in a bi-fold orientation, and
wherein the opening in the sheath is located between the wing-tip portion of the first wing and the wing-base portion of the second wing.

12. The delivery system of claim 10, wherein:
the balloon is a dual-wing balloon having first and second wings, each wing having a respective wing-tip portion and a wing-base portion, and
wherein the balloon is wrapped about the catheter in a U-fold orientation, and
wherein the opening in the sheath is located between the wing tip of the first wing and the wingtip of the second wing.

13. The delivery system of claim 10, wherein the balloon is a tri-wing balloon having three wings, each wing having a respective wingtip portion and wing base portion,
wherein the balloon is wrapped about the catheter such that a wingtip portion of a first wing is folded toward a wingbase portion of a next adjacent wing, and
wherein the opening in the sheath is located between the wingtip portion of the first wing and the wingbase portion of the next adjacent wing.

14. The delivery system of claim 13, wherein the opening in the sheath is located within 20% of a midpoint between the wingtip portion of the first wing and the wingbase portion of the next adjacent wing.

15. A system, comprising:
a catheter having a distal end and a proximal end;
a balloon, in a deflated condition, positioned at the distal end of the catheter and wrapped about the distal end of the catheter, the balloon comprising at least two wing portions;
a medical device, having a compressed state and an expanded state, positioned about the balloon; and
a sheath, comprising sheath material, positioned about the medical device to hold the medical device in the compressed state, the sheath material comprising a predetermined sheath portion,
wherein the predetermined sheath portion is located at a position as a function of positions of the at least two wing portions of the balloon.

16. The system of claim 15, wherein the balloon is one of:
a bi-wing structure with only two wings; and
a tri-wing structure with three wings.

17. The system of claim 15, wherein the predetermined sheath portion comprises at least one of:
a slit in the sheath material;
a hole in the sheath material; and
a weakened portion of the sheath material.

18. The system of claim 17, wherein the predetermined sheath portion comprises the weakened portion of the sheath material, and
wherein the weakened portion of the sheath material is the result of at least one of:
chemical application; and
mechanical stress.

19. The system of claim 15, wherein the balloon is of a bi-wing structure with only first and second wings, each wing having respective base and tip portions, the system further comprising:
the first wing of the balloon circumferentially wrapped in a first direction around the distal end of the catheter, the tip portion of the first wing oriented toward the base portion of the second wing and
the second wing of the balloon circumferentially wrapped in the first direction around the distal end of the catheter, the tip portion of the second wing oriented toward the base portion of the first wing,
wherein the predetermined sheath portion is one of:
located between the tip portion of the first wing and the base portion of the second wing; and
located between the tip portion of the second wing and the base portion of the first wing.

20. The system of claim 19, wherein the predetermined sheath portion comprises at least one of:
a slit in the sheath material;
a hole in the sheath material; and
a weakened portion of the sheath material.

21. The system of claim 20, wherein the predetermined sheath portion comprises the weakened portion of the sheath material, and
wherein the weakened portion of the sheath material is the result of at least one of:
chemical application; and
mechanical stress.

22. The system of claim 15, wherein the balloon is of a bi-wing structure with first and second wings, each wing having respective base and tip portions, the system further comprising:
the first wing circumferentially wrapped in a first direction around the distal end of the catheter; and
the second wing circumferentially wrapped in a second direction, opposite the first direction, around the distal end of the catheter,
wherein the predetermined sheath portion is located between the tip portion of the first wing and the tip portion of the second wing.

23. The system of claim 22, wherein the predetermined sheath portion comprises at least one of:
a slit in the sheath material;
a hole in the sheath material; and
a weakened portion of the sheath material.

24. The system of claim 23, wherein the predetermined sheath portion comprises the weakened portion of the sheath material, and
wherein the weakened portion of the sheath material is the result of at least one of:
chemical application; and
mechanical stress.

25. The system of claim 15, wherein the balloon is of a tri-wing structure with first, second, and third wings, each wing having respective base and tip portions, the system further comprising:
the first wing circumferentially wrapped in a first direction around the distal end of the catheter;
the second wing circumferentially wrapped in the first direction around the distal end of the catheter; and
the third wing circumferentially wrapped in the first direction around the distal end of the catheter,
wherein the predetermined sheath portion of the sheath material is one of:
located between the tip portion of the first wing and the base portion of the second wing;
located between the tip portion of the second wing and the base portion of the third wing; and
located between the tip portion of the third wing and the base portion of the first wing.

26. The system of claim 25, wherein the predetermined portion comprises at least one of:
a slit in the sheath material;
a hole in the sheath material; and
a weakened portion of the sheath material.

27. The system of claim 26, wherein the predetermined sheath portion comprises the weakened portion of the sheath material, and
wherein the weakened portion of the sheath material is the result of at least one of:
chemical application; and
mechanical stress.

28. A method of providing an ostial protection device delivery system, the method comprising:
providing a catheter having a distal end and a proximal end;
positioning a deflated balloon at the distal end of the catheter, the balloon comprising wing portions;
wrapping the wing portions about the distal portion of the catheter so as to facilitate inflation of the balloon;
providing an ostial protection device about the deflated balloon;
positioning an elongate tubular sheath, the sheath having a proximal end and a distal end oriented with the catheter, the sheath comprising a predefined sheath portion, about the ostial protection device and the balloon to hold the ostial protection device in a compressed state; and
orienting the predefined sheath portion in a predetermined relationship as a function of locations of the folded wing portions of the balloon.

29. The method of claim 28, wherein the balloon is of a bi-wing structure with only first and second wings, each wing having respective base and tip portions, the method further comprising:
wrapping the first wing of the balloon circumferentially in a first direction around the distal portion of the catheter, the tip portion of the first wing oriented toward the base portion of the second wing;
wrapping the second wing of the balloon circumferentially in the first direction around the distal portion of the catheter tip portion, the second wing oriented toward the base portion of the first wing,
locating the predefined sheath portion as one of:
between the tip portion of the first wing and the base portion of the second wing; and
between the tip portion of the second wing and the base portion of the first wing.

30. The method of claim 29, wherein providing the predefined sheath portion comprises at least one of:
slitting the sheath material;
creating a hole in the sheath material; and
creating a weakened portion of the sheath material.

31. The method of claim 30, wherein creating the weakened portion comprises at least one of:
applying a chemical substance to the sheath material; and
applying a mechanical stress to the sheath material.

32. The method of claim 28, wherein the balloon is of a bi-wing structure with first and second wings, each wing having respective base and tip portions, the method further comprising:
wrapping the first wing circumferentially in a first direction around the distal portion of the catheter;
wrapping the second wing circumferentially in a second direction, opposite the first direction, around the distal portion of the catheter; and
locating the predefined sheath portion between the tip portion of the first wing and the tip portion of the second wing.

33. The method of claim 32, wherein providing the predefined sheath portion comprises at least one of:
slitting the sheath material;
creating a hole in the sheath material; and
creating a weakened portion of the sheath material.

34. The method of claim 33, wherein creating the weakened portion comprises at least one of:
applying a chemical substance to the sheath material; and
applying a mechanical stress to the sheath material.

35. The method of claim 28, wherein the balloon is of a tri-wing structure with first, second, and third wings, each wing having respective base and tip portions, the method further comprising:
wrapping the first wing circumferentially in a first direction around the distal portion of the catheter;
wrapping the second wing circumferentially in the first direction around the distal portion of the catheter;
wrapping the third wing circumferentially in the first direction around the distal portion of the catheter; and
locating the predefined sheath portion as one of:
between the tip portion of the first wing and the base portion of the second wing;
between the tip portion of the second wing and the base portion of the third wing; and
between the tip portion of the third wing and the base portion of the first wing.

36. The method of claim 35, wherein providing the predefined sheath portion comprises at least one of:
slitting the sheath material;
creating a hole in the sheath material; and
creating a weakened portion of the sheath material.

37. The method of claim 36, wherein creating the weakened portion comprises at least one of:
applying a chemical substance to the sheath material; and
applying a mechanical stress to the sheath material.
